(19) United States
(12) Patent Application Publication
(10) Pub. No.: US 2012/0321584 A1
(43) Pub. Date: Dec. 20, 2012

(54) PROCESSES FOR PREPARING AMINE SALTS OF KMUP-3 AND USE THEREOF

(75) Inventor: Ing-Jun Chen, Kaohsiung (TW)

(73) Assignee: KAOHSIUNG MEDICAL UNIVERSITY, Kaohsiung (TW)

(21) Appl. No.: 13/356,039

(22) Filed: Jan. 23, 2012

(30) Foreign Application Priority Data
Jun. 14, 2011 (TW) 100120778

(51) Int. Cl. A61K 31/785; C08G 69/36; C08B 37/00; C08B 37/02; C08B 37/10; A61P 9/00; C08G 63/08; A61K 31/717; A61K 31/728; A61K 31/721; A61K 31/727; A61P 9/04; C08B 15/06; C08F 220/06

(52) U.S. Cl. 424/78.29; 536/31; 525/420; 536/55.1; 536/51; 536/21; 525/329.9; 525/437; 514/57; 514/54; 514/59; 514/56

(57) ABSTRACT

An inhibiting heart failure disease pharmaceutical composition is provided.
The pharmaceutical composition includes:
an effective amount of a KMUP-3 amine salt of formula (I);

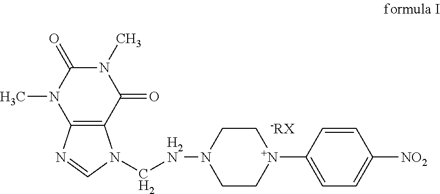

wherein RX contains a carboxylic group donated from one selected from a group consisting of a sodium carboxyl methylcellulose (sodium CMC), a γ-polyglutamic acid derivative, and co-polymer salt;
RX⁻ is an anion form of the carboxylic group; and
a pharmaceutically accepted carrier.

The application claims the benefit of Taiwan Patent Application No. 100120778, filed on Jun. 16, 2011, in the Taiwan Intellectual Property Office, the disclosures of which are incorporated herein in their entirety by reference.

### FIELD OF THE INVENTION

The present invention of KMUP amine salt related to and therapeutically used for the treatment of heart failure by increasing cardiac output via RhoA-mediated Ca²⁺ sensitization, under Ca²⁺ entry protection via increased PKG and ancillary vasodilation via activation of eNOS.

### BACKGROUND OF THE INVENTION

Cardioprotection and vasorelaxation are both required to prevent pressure-overloading in patients of congestive heart failure (CHF) with hypoperfused myocardium. Therefore, treatment with vasodilatory NO/cGMP-enhancer characterized by having Ca²⁺ sensitization activity, ably increasing cardiac output, would be better for CHF. KMUP-1, an analogue of KMUP-3, has displayed NO/cGMP-dependent inhibition of Rho kinase (ROCK) and inhibited β-adrenoceptor (β-AR) agonist-induced chronic cardiac hypertrophy. These results encouraged us to examine the RhoA-mediated Ca²⁺ sensitization activity of KMUP-3 for the treatment of heart failure (Endoh M., 2007). This study investigates whether KMUP-3, a PDE-3/PDE-4/PDE-5 inhibitor, energy-economically increasing left ventricular systolic blood pressure (LVSP) and atria inotropy, is more therapeutically relevant than each class of PDE inhibitors for CHF patients with hypoperfused myocardium. Since RhoA/ROCK mediates downstream Ca²⁺ sensitization signaling via G-protein coupled receptors (GPCRs), we wonder that wide range cardiac PDE-3/PDE-4/PDE-5 inhibition by KMUP-3 involving activation of RhoA/ROCK and Ca²⁺ sensitization activity via GPCRs.

Over stimulation of cardiac myocytes by cAMP/Ca²⁺ is associated with excessive workload, which increases mortality of CHF patients, thus Ca²⁺-sensitization, attributable to protein kinases (PKs), including PKA, PKCα and RhoA/ROCK expression, the upstream signaling of myosin light chain phosphatase (MLCP), requires protection strategy to prevent from cardiac-overstimulation of CHF and exacerbation of cardiac hypertrophy. This realization has caused a paradigm shift in the pharmacotherapy of CHF from cardiotonic to cardioprotective therapy. Ca²⁺ sensitization by calcium binding to troponin has been allowed to treat CHF with levosimendan. In contrast, our study is to expose cardiac Ca²⁺ sensitization by activating intracellular RhoA/ROCK through agonist activity on cardiac cell membrane GPCRs.

PDE-3 selective inhibitors have been used to treat heart failure (HF) as a replacement for digitalis. They improve the hemodynamic status in HF via inotropic/vasodilatory activity attributed to cAMP. In contrast, PDE-3/PDE-5 inhibitor inhibits heart failure via cGMP. Moreover, PDE-3/PDE-4 inhibition enforces cardiac muscle contractility by increasing cAMP, easily initiating cardiac-overstimulation. Combination therapy with β-AR blocker and PDE-3 inhibitor has prolonged the survival of patients with CHF and cGMP enhancers have prevented β-AR agonist-induced cardiac hypertrophy.

Inotropy and L-type Ca²⁺ currents are controlled by PDE-3/PDE-4 through activating β-ARs. Compared to PDE-3/PDE-4 inhibitor, KMUP-3's additional PDE-5A inhibition and cGMP-enhancing activity should have greater cardioprotective benefits. Acute elevation of cGMP usually exerts negative metabolic/inotropic effects, whereas elevation of cGMP prevents cardiac hypertrophy. These considerations led us to search for NO/cGMP-enhancing PDE inhibitors, characterized by having β-AR blocker-like activity. Inhibition of PDE-5A, blunting the stimulation of β-AR, is associated with cGMP-enhancement and has shown benefits in cardiac hypertrophy.

RhoA is a small GTP-binding protein controlled by guanine exchange factors, which catalyze the exchange of GDP for GTP. RhoA/ROCK is involved in cardiac contraction and may be an interesting drug target. ROCK has also been shown to cause cardiac hypertrophy by over workload. Although there are much evidences that activation of RhoA is deleterious to the heart, RhoA may have the capacity to confer protection of cardiomyocytes by signaling through Akt. Theoretically, Ca²⁺ sensitization by RhoA/ROCK pathway to increase inotropy does not need much intracellular Ca²⁺, in contrast to the requirements for Ca²⁺ binding to troponin.

GPCR agonists are composed of flexible structures and difficult to specifically evaluate. To date, GPCRs has been activated by thrombin and lysophosphatidic acid in non-cardiac cells characterized by Ca²⁺ entry property. There are lack mentions about GPCRs agonists showing Ca²⁺ entry blockade in the heart. To examine the benefits of cardioprotective GPCRs agonists in CHF, hemodynamic activity, Ca²⁺ entry, protein kinases (PKs) and RhoA/ROCK expression, Ca²⁺ sensitization and their regulation by cGMP/cAMP were investigated in this study.

Neovascularization, involving arteriogenesis, angiogenesis, or vascularization, is an important compensatory response to atherosclerotic obstructive arterial disease, but natural mechanisms rarely restore normal or near normal maximal flow capacity. Multiple protein, gene, and cell-based strategies have improved collateral flow in ischemic animal models. Nonetheless, clinical trials have not yielded promising results. Undoubted contributors to the disparities are the numerous risk factors present in patients but not in the experimental animals. One of these risk factors is age—i.e, clinical interventions are targeted to an older population, whereas the experimental animals have invariably been young.

The present invention provides KMUP amine salt synthesized by the KMUP compound and a carboxylic acid derivative of one selected from a group consisting of a statin, a non-steroid anti-inflammatory (NSAIDs) and an anti-asthmatic drug. The pharmaceutical composition for a treatment of an interstitial lung disease have applied as Ser. No. 11/857,483 filed on Sep. 19, 2007.

### SUMMARY OF THE INVENTION

In accordance with an aspect of the present invention, an inhibiting heart failure disease complex compound is provided. The complex compound comprising a structure being one of formula (I),

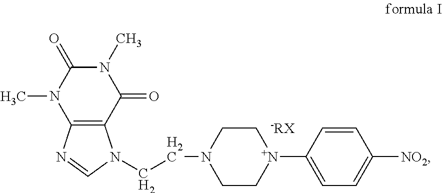

Wherein RX contains a carboxylic group which donated from one of a sodium carboxyl methylcellulose (sodium CMC), a γ-polyglutamic acid derivative and a co-polymer salt; and
RX⁻ substituent is an anion of the carboxylic group carrying a negative charge.

In accordance with another aspect of the present invention, an inhibiting heart failure disease pharmaceutical composition is provided. The pharmaceutical composition includes:

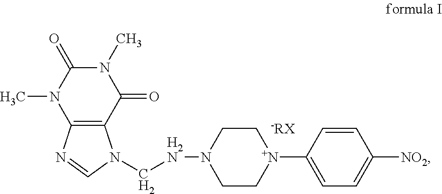

an effective amount of a compound of formula (I), wherein
RX contains a carboxylic group which donated from one of a sodium carboxyl methylcellulose, a γ-polyglutamic acid derivative and a co-polymer salt; and
RX⁻ substituent is an anion of the carboxylic group carrying a negative charge; and
a pharmaceutically acceptable carrier.

In accordance with an aspect of the present invention, an improving cardiac output dose-dependently under Ca²⁺ entry protection complex compound is provided. There is also provided a another aspect of the present invention, preventing or managing of heart failure and/or connected diseases therewith is selected from the group consisting of acute and chronic cardiac insufficiency, arterial hypertension, coronary heart disease, stable and unstable angina pectoris, myocardial ischemia, myocardial infarction, shock, arteriosclerosis, atrial and ventricular arrhythmias, transitory and ischemic attacks, stroke, inflammatory cardiovascular diseases, peripheral and cardiac vascular diseases, peripheral circulation disorders, spasms of the coronary arteries and peripheral arteries, thromboses, thromboembolic diseases, edema formation such as for example pulmonary edema, cerebral edema, renal edema or cardiac insufficiency-related edema, and restenosis for example after thrombolysis treatments, percutaneous-transluminal angioplasties (PTA), transluminal coronary angioplasties (PTCA), heart transplants and bypass operations. The complex compound comprising a structure being one of formula (I),

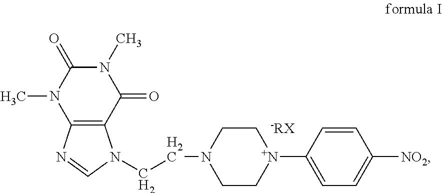

Wherein RX contains a carboxylic group which donated from one of a sodium carboxyl methylcellulose, a γ-polyglutamic acid derivative and a Co-polymer salt; and
RX⁻ substituent is an anion of the carboxylic group carrying a negative charge.

In accordance with a further aspect of the present invention, an improving cardiac output dose-dependently under Ca²⁺ entry protection pharmaceutical composition is provided. The pharmaceutical composition includes:

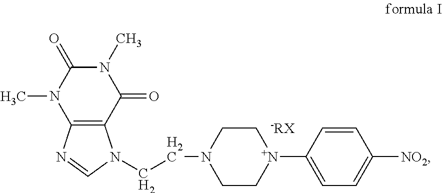

an effective amount of a compound of formula (I), wherein
RX contains a carboxylic group which donated from one of a sodium carboxyl methylcellulose, a γ-polyglutamic acid derivative and a Co-polymer salt; and
RX⁻ substituent is an anion of the carboxylic group carrying a negative charge; and
a pharmaceutically acceptable carrier.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 A-1 B show hemodynamic effects of KMUP-3. Rats were treated with KMUP-3 (0.03˜0.075 mg/kg, i.v., 30 min and 0.03˜0.1 mg/kg, i.p.).

FIG. 1 A shows the recording traces of LVSP.

FIG. 1 B shows the recording traces of MABP.

FIG. 2 A-2 B show LVSP, dp/dt and MABP. KMUP-3-induced effects were inhibited by pretreatment with Y27632 or ketanserin.
+Y27632 indicated that injections of KMUP-3 (1 mg/kg) and Y27632 (1 mg/kg);
+ketanserin indicated that injections of KMUP-3 (1 mg/kg) and ketanserin (1 mg/kg).
Values are expressed as means±SE of n=6. * indicated P<0.05 and ** indicated P<0.01

FIG. 2 A shows the increase percentage of LVSP.

FIG. 2 B shows the increase percentage of dp/dt.

FIG. 2 C shows the increase percentage of MABP.

FIG. 3 A-3 B show the effect of intravenous perfusion of KMUP-3

FIG. 3 A shows change in MABP.

FIG. 3 B shows change in HR.

FIG. 4 A-4 B show the effect of intra-peritoneal injection of KMUP-3

FIG. 4 A shows the change in MABP.

FIG. 4 B shows the change in HR.

FIG. 5 A-5 B show the effect of spontaneously beating right atria.

FIG. 5 A shows the change of contraction force percentage.

FIG. 5 B shows the effect of frequency percentage.

FIG. 6 shows the effect of frequency percentage of KMUP-3, atropine and 7-nitroindazole. Values are expressed as means±SE, n=6. *P<0.05 versus KMUP-3.

FIG. 7 shows the change of contraction force percentage. *P<0.05; **P<0.01 versus KMUP-3

FIG. 8 shows the effects of pretreatment with SQ22536 or ODQ on KMUP-3-induced electronically-stimulated left atria contraction. *P<0.05; **P<0.01 versus KMUP-3.

FIG. 9 shows the effects of pretreatment with 7-nitroindazole on KMUP-3-induced left atria contraction. **P<0.01 versus control; ^{#}P<0.01 versus KMUP-3.

FIG. 10 A-10 B show the change of contraction force percentage.

FIG. 10 A shows the effects of pretreatment with Y27632, exoenzyme C3 and L-NAME. **P<0.01 versus KMUP-3,

FIG. 10 B shows the effects of pretreatment with, propranolol and ketanserin. **P<0.01 versus KMUP-3.

FIG. 11 A-11 C show the protein expression of RhoA and ROCKII

FIG. 11 A shows the protein expression of RhoA

FIG. 11 B shows the protein expression of ROCKII

FIG. 11 C shows the RhoA activation by KMUP-3 in neonatal myocytes

FIG. 12 A-12 B show the translocation of PKCα and the phosphorylation of CPI-17. Values are expressed as means±SE of 6. *P<0.05; **P<0.01 versus control.

FIG. 12 A the percentage of total PKCα expression

FIG. 12 B shows the CPI-17 phosphorylation

FIG. 13 A-13 C show the effects of Y27632 on KMUP-3-induced PKs expression. Values are expressed as means±SE of n=6. **P<0.01; ***P<0.001 versus control; ^{##}P<0.01 versus KMUP-3

FIG. 13 A shows the expression of ROCKII.

FIG. 13 B shows the expression of PKA.

FIG. 13 C shows the expression of PKG.

FIG. 14 A-14 B show the effects of KMUP-3 on expression of eNOS/p-NOS. Values are expressed as means±SE of n=6. *P<0.05; **P<0.01 versus control; ^{##}P<0.01 versus KMUP-3.

FIG. 14 A shows KMUP-3 increased eNOS

FIG. 14 B shows KMUP-3 increased p-NOS

FIG. 15 A-15 B show KMUP-3 increased cAMP and cGMP. Values are expressed as means±SE of n=6. *P<0.05; **P<0.01 versus control; ^{##}P<0.01 versus KMUP-3.

FIG. 15 A shows the change of cAMP.

FIG. 15 B shows the change of cGMP.

FIG. 16 shows RhoA activation of neonatal myocytes. Values are expressed as means±SE of n=6. *P<0.05; **P<0.01 versus control.

FIG. 17 A-17 B show the expression of ROCK and eNOS in the vascular system. Values are expressed as means±SE of n=6. *P<0.05; **P<0.01 versus control.

FIG. 17 A shows ROCK and eNOS-expression of rat aorta.

FIG. 17 B shows ROCK and eNOS-expression of pulmonary artery (PA).

FIG. 18 A-18 B show the effects of KMUP-3 on Ca²⁺ entry currents in rat heart-derived H9c2 cells. Values are expressed as means±SE of n=6. *P<0.05 versus control; ^{#}P<0.05 versus KMUP-3.

FIG. 18 A shows the recording trace Ca²⁺ entry currents of KMUP-3.

FIG. 18 B shows decreased the inward currents of KMUP-3.

FIG. 19 A-19 B show the effects of KMUP-3 combination with the PKG antagonist KT5823 on Ca²⁺ entry currents. Values are expressed as means±SE of n=6. *P<0.05 versus control; ^{#}P<0.05 versus KMUP-3.

FIG. 19 A shows the recording trace Ca²⁺ entry currents of KMUP-3 combination with KT5823.

FIG. 19 B shows decreased the inward currents of KMUP-3 combination with KT5823.

FIG. 20 A-20 B show the effects of KMUP-3 combination with the PKG antagonist Y27632 on Ca²⁺ entry currents. Values are expressed as means±SE of n=6. *P<0.05 versus control; ^{#}P<0.05 versus KMUP-3.

FIG. 20 A shows the recording trace Ca²⁺ entry currents of KMUP-3 combination with Y27632.

FIG. 20 B shows decreased the inward currents of KMUP-3 combination with KT5823.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

A KMUP-3 amine salt having a formula (I),

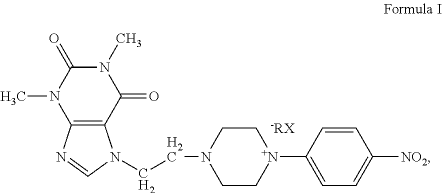

wherein RX contains a carboxylic group which donated from one of a sodium carboxyl methylcellulose (sodium CMC), a γ-polyglutamic acid derivative and a co-polymer salt; and
RX⁻ may be an anion of the above-mentioned groups carrying a negative charge.

Preferably, γ-Polyglutamic acid derivative being one selected from a group consisting of a sodium polyglutamate, an alginate-poly-lysine-alginate (APA) and a calcium polyglutamate-alginate sodium. Co-polymer salts includes one selected from a group consisting of a hyaluronic acid, a polyacrylic acid, a dextran sulfate, a heparan sulfate, a polylactic acid or polylactide (PLA), a polylactic acid sodium (PLA sodium) and a polyglycolic acid sodium (PGA sodium).

A pharmaceutical composition is provided in the present, in which the active agent is a theophylline-based moiety compound for treating an ischemia disease, and increases cardiac output via RhoA-mediated Ca²⁺ sensitization, under Ca²⁺ entry protection, useful for improving heart failure.

Preferably, a theophylline-based moiety compound derivative, i.e. KMUP-3, which is obtained by reacting theophylline compound with piperazine compound and then recrystallizing the intermediate therefrom, is provided in the present invention.

To achieve the above purpose, KMUP-3 amine salt (formula I) can be synthetically produced from the 2-chloroethyltheophylline compound and piperazine substituted compound.

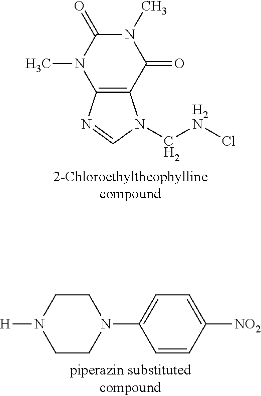

The compounds of KMUP-3 amine salt set forth in the examples below were prepared using the following general procedures as indicated. 2-Chloroethyl theophylline, NaOH and 4-nitrobenzenepiperazin are dissolved in hydrous ethanol, and then heated under reflux. Allowed to stand overnight, the cold supernatant was decanted for proceeding, efficient removal of solvents by vacuum concentration, and then the residue were dissolved with one-fold volume of ethanol and a sufficiently volume of 2N hydrochloric acid (HCl), kept at warmer temperature, to make a saturated solution (pH 1.2). The saturated solution was sequentially treated, decolorized with activated charcoal, filtered, deposited overnight and filtered to obtain KMUP-3 HCl with a white crystal.

KMUP-3 is dissolved in a mixture of ethanol and polyglutamate. The solution is reacted at warmer temperature, the methanol is added thereinto under room temperature, and the solution is incubated over night for crystallization and filtrated to obtain KMUP-3-polyglutamate salt.

The formation I of KMUP-3 amine salt has been carried out in the presence of KMUP-3 compound with the group of sodium carboxyl methylcellulose (CMC), γ-polyglutamic acid derivative or co-polymer salt, followed by the synthesis process.

Preferably, in one embodiment, the compound of formula I is KMUP-3 amine salt, which has the chemical name RX-7-[2-[4-(4-nitrobenzene)piperazinyl]ethyl]-1,3-dimethylxanthine (i.e. RX-KMUP-3 salt). RX contains a carboxylic group which donated from a group consisting of a member of sodium carboxyl methylcellulose, γ-polyglutamic acid derivative and co-polymer salt; RX⁻ may be an anion of the above-mentioned groups carrying a negative charge.

Specifically speaking, KMUPs amine salt compounds in one embodiment, as KMUP-3-CMC salt, KMUP-3-polyglutamate, KMUP-3-APA, KMUP-3-calcium polyglutamate-alginate, KMUP-3-hyaluronic acid salt, KMUP-3-polyacrylic acid salt, KMUP-3-heparan salt, KMUP-3-polylactide salt, KMUP-3-dextran acid salt, etc.

In accordance with a further aspect of the present invention, depending on the desired clinical use and the effect, the adaptable administration method of pharmaceutical composition includes one selected from a group consisting of an oral administration, an intravenous injection, a subcutaneous injection, an intraperitoneal injection, an intramuscular injection and a sublingual administration.

The term excipients or “pharmaceutically acceptable carrier or excipients” and “bio-available carriers or excipients” mentioned above include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself. Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human. A therapeutic effect can be achieved by using the dosage form in the present invention by the local or sublingual administration via the venous, oral, and inhalation routes or via the nasal, rectal and vaginal routes. About 0.1 mg to 1000 mg per day of the active ingredient is administered for the patients of various diseases.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylmethyl cellulose or the analogous dispersing agent. Other carriers are common surfactant such as Tween and Spans or other analogous emulsion, or the pharmaceutically acceptable solid, liquid or other bio-available enhancing agent used for developing the formulation that is used in the pharmaceutical industry.

The composition for oral administration adopts any oral acceptable formulation, which includes capsule, tablet, pill, emulsion, aqueous suspension, dispersing agent and solvent. The carrier is generally used in the oral formulation. Taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The compound of the present invention can also be administered intravenously, as well as subcutaneously, parentally, muscular, or by the intra-articular, intracranial, intra-articular fluid and intra-spinal injections, the aortic injection, the sterna injection, the intra-lesion injection or other appropriate administrations.

Table 2 shows the changes of protein expression of eNOS, and ROCKII represented by optical density (%) after application of KMUPs salts (10 μM) for 120 min, compared to the control without treatment.

TABLE 2

| KMUP-3's | eNOS | PDE-3 | PDE-4 | PDE-5 | ROCKII |
|---|---|---|---|---|---|
| Vehicle | 100 (%) | 100 (%) | 100 (%) | 100 (%) | 100 (%) |
| —HCl | 138 ± 5.2 | 55 ± 2.5 | 48 ± 2.1 | 48 ± 1.9 | 42 ± 2.2 |
| —APA | 131 ± 5.5 | 56 ± 2.3 | 52 ± 2.1 | 54 ± 1.7 | 38 ± 2.8 |
| —Sod•Pga | 133 ± 3.3 | 60 ± 1.8 | 55 ± 2.3 | 62 ± 1.4 | 54 ± 3.8 |
| —PLA | 143 ± 3.1 | 47 ± 1.8 | 45 ± 2.7 | 43 ± 1.9 | 52 ± 2.7 |
| -hyaluronic | 147 ± 5.5 | 58 ± 2.6 | 54 ± 2.5 | 58 ± 1.6 | 48 ± 3.6 |
| acid | | | | | |
| -dextran | 148 ± 2.8 | 48 ± 1.8 | 52 ± 2.7 | 53 ± 1.9 | 43 ± 3.6 |
| sulfate | | | | | |

(note)
Vehicle = Without treatment control
KMUP-3's = KMUP-3-HCl, KMUP-3-hyaluronic acid, KMUP-3-APA, KMUP-3-sodium polyglutamate, KMUP-3-PLA, KMUP-3-dextran sulfate,
APA = alginate-poly-lysine-alginate
Sod•Pga = sodium polyglutamate
PLA = polylactic acid

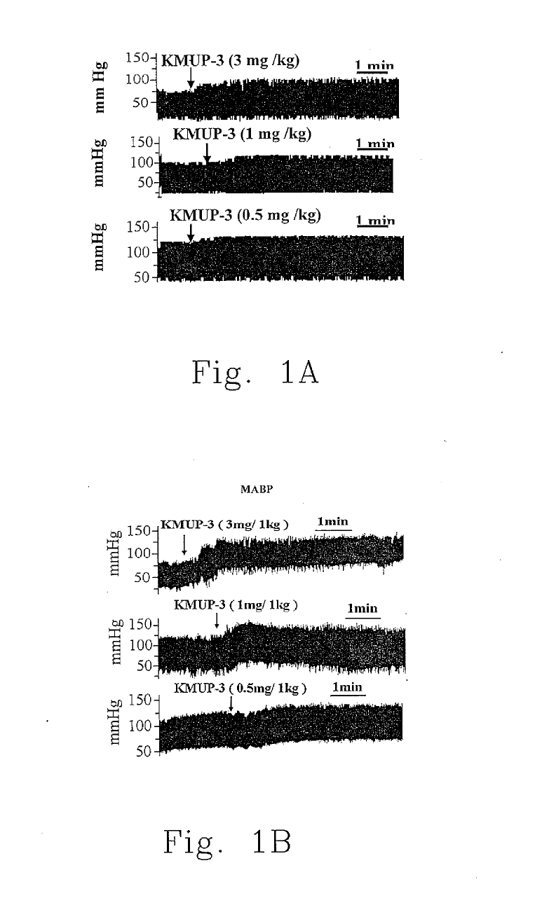

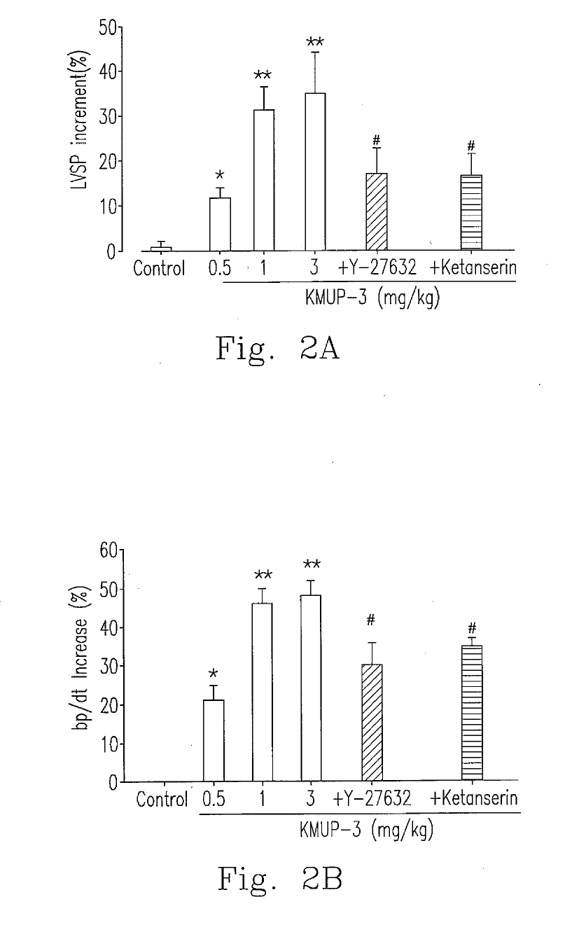

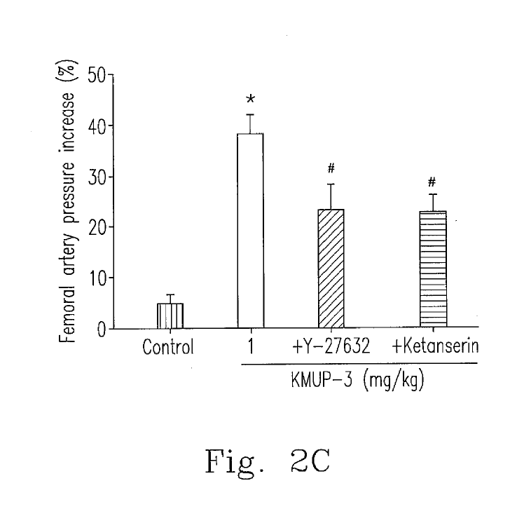

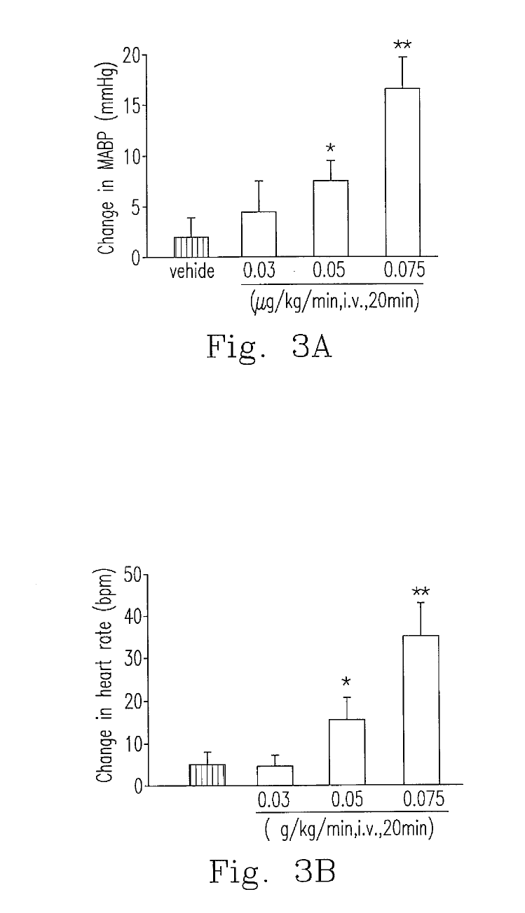

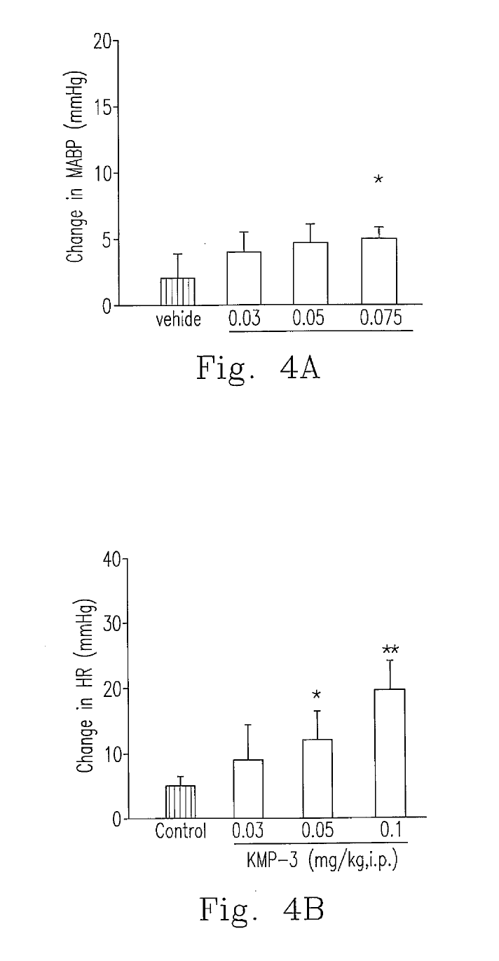

LVSP, dP/dt, MABP and HR. As shown by the trace recording in FIG. 1 A-4 B, intravenous injection of KMUP-3 (0.5-3 mg/kg, i.v.) within 2 min dose-dependently increased the LVSP (left ventricular systolic pressure), dP/dt and MABP (mean artrial blood pressure) in anesthetized rats. Pretreatment with intravenous Y27632, ketanserin and propranolol at 1 mg/kg respectively inhibited the effects induced by KMUP-3 (1 mg/kg, i.v.). Intravenous perfusion of KMUP-3 (0.03-0.075 μg/kg/min×20 min) dose-dependently increased MABP and HR (FIG. 3 A and FIG. 3 B). Intra-peritoneal injection of KMUP-3 (0.03-0.1 mg/kg) dose-dependently increased MABP and HR (FIG. 4 A and FIG. 4 B).

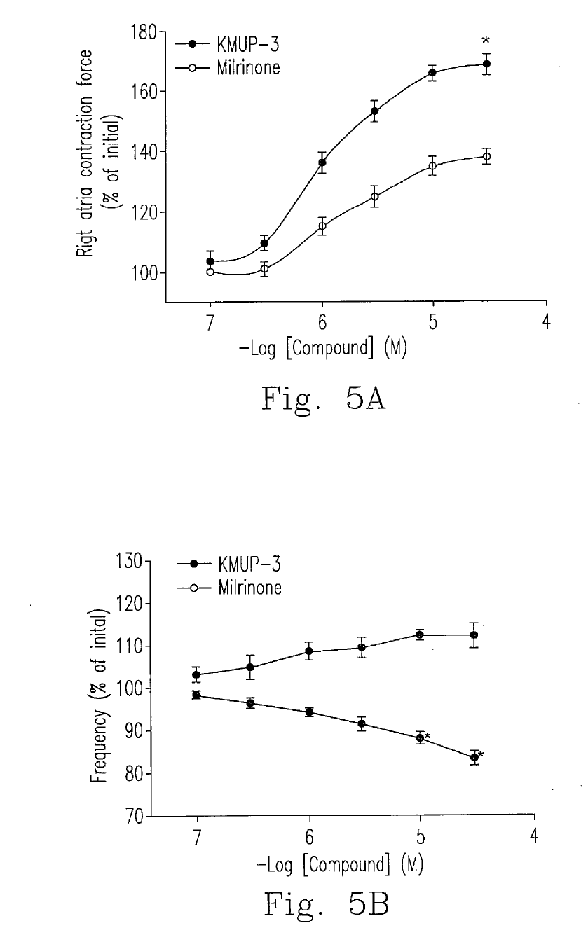

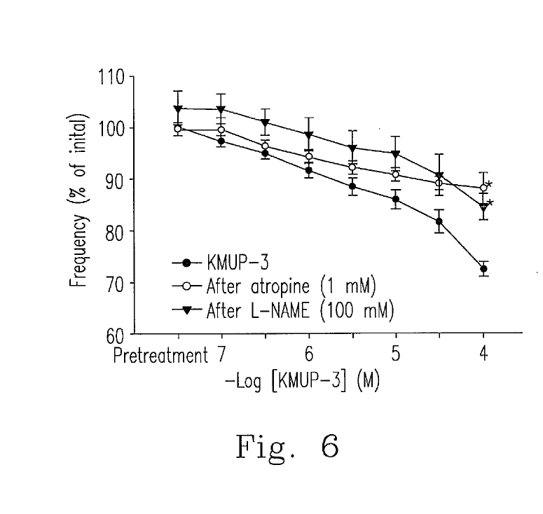

Spontaneously beating right atria. As shown in FIG. 5 A and FIG. 5 B, KMUP-3 (0.1-30 μM) concentration-dependently increased inotropic and decreased chronotropic effects in isolated spontaneously beating right atrium. In contrast, milrinone (0.1˜30 μM) increased both inotropic and chronotropic activities. The beating frequency decreased by KMUP-3 was up-regulated by pretreatment with atropine (1 μM) and 7-nitroindazole (100 μM) (FIG. 6).

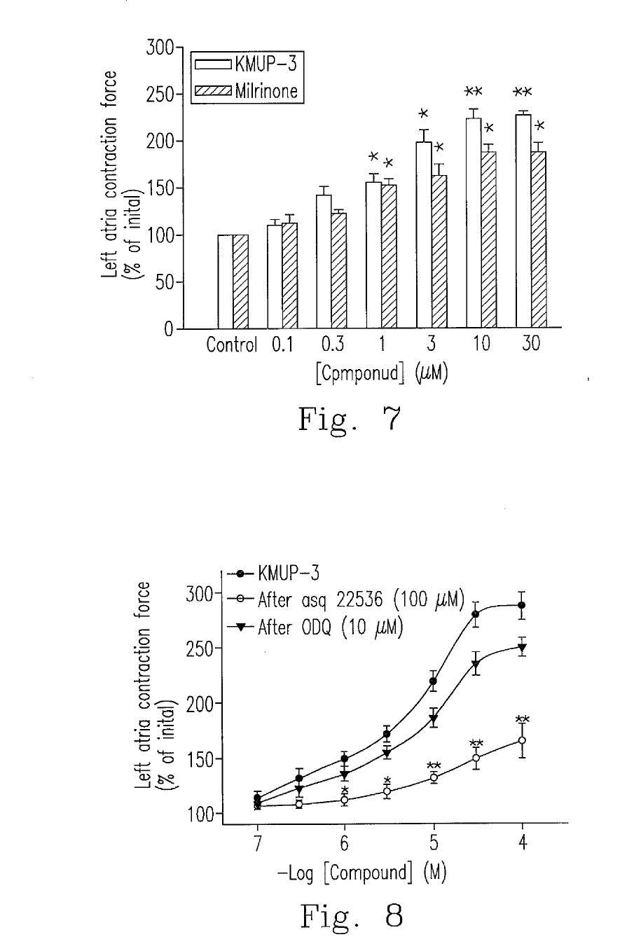

Electrically-stimulated left atria. The inotropic effects of KMUP-3 and milrinone were studied under electrical stimulation (1 Hz) at a constant rate. As shown in FIG. 7, both KMUP-3 and milrinone (0.1˜30 μM) concentration-dependently increased left atrium contractility. KMUP-3 was more potent than milrinone for increasing contractility (P<0.05).

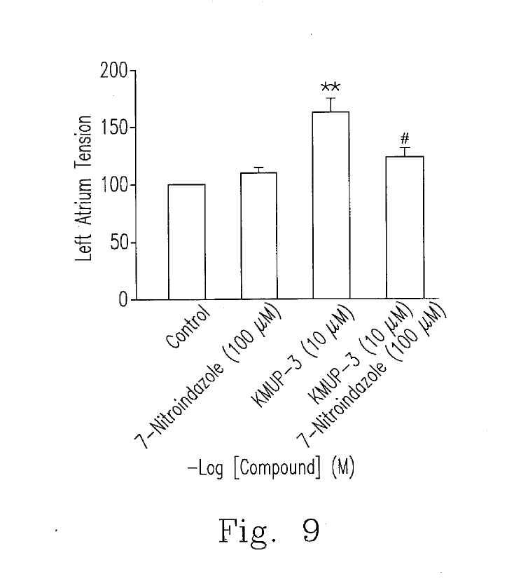

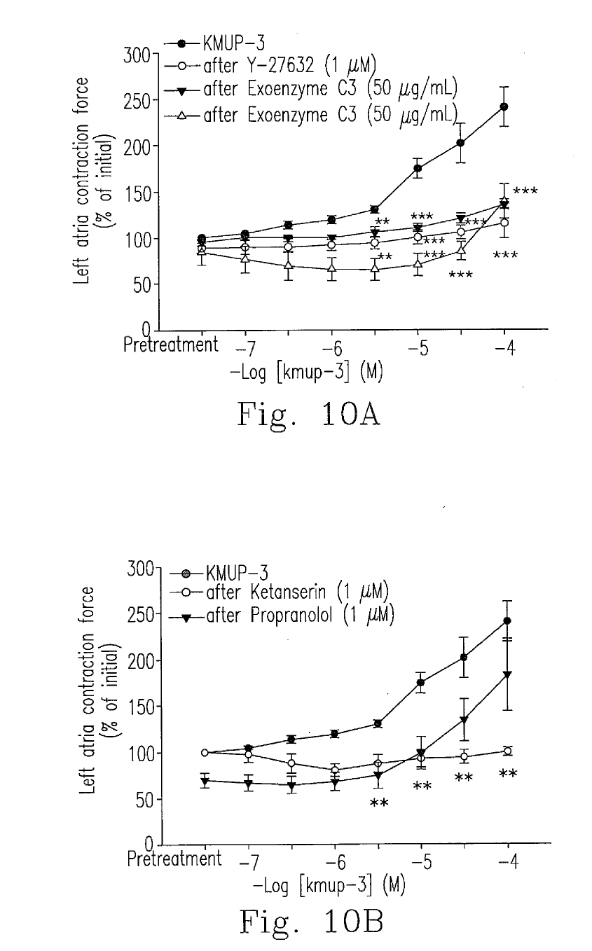

Pretreatment with the sGC inhibitor ODQ and the eNOS inhibitor L-NAME inhibited KMUP-3-induced contractility, suggesting the involvement of the NO/sGC/cGMP pathway (FIG. 8). KMUP-3 (10 μM) increased the left atria contractility and was reduced by combination with 7-nitroindazole (100 μM) (FIG. 9). Pretreatment with propranolol and Y27632 at 1 μM, C3 exoenzyme (50 μg/mL), ketanserin (1.0 μM) and L-NAME (100 μM) all reduced KMUP-3-induced positive inotropic activity in left atria (FIG. 10 A and FIG. 10B).

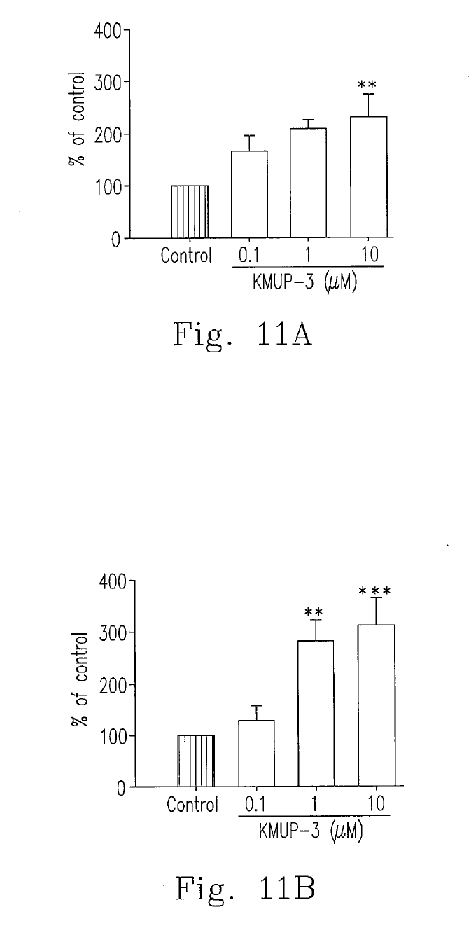

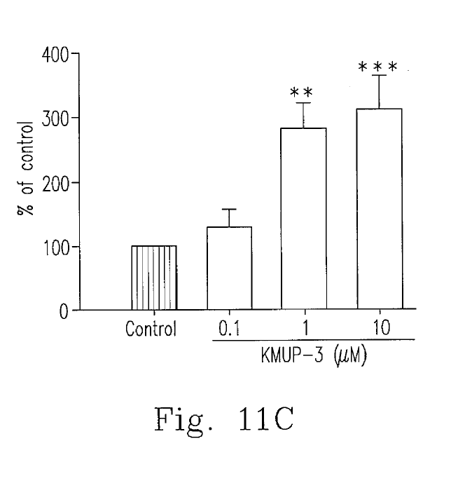

RhoA/ROCKII and PKA. RhoA, ROCKII and PKA expression were significantly increased by KMUP-3 (0.1-10 μM) in electrically-stimulated left guinea-pig atria (FIG. 11A, 11B, and FIG. 11C).

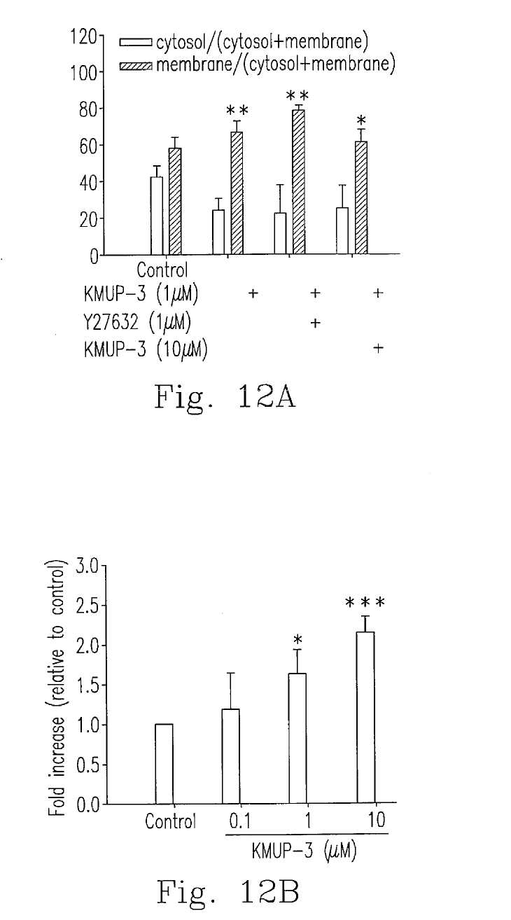

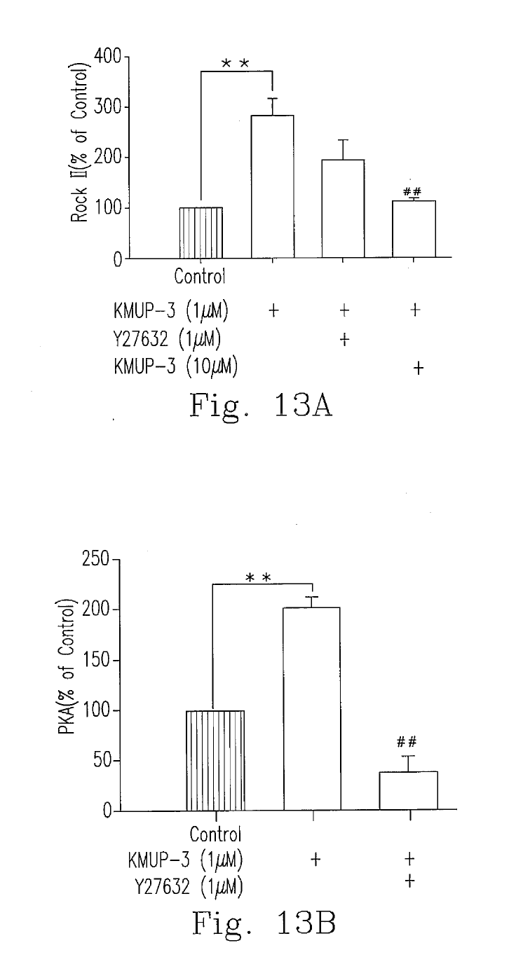

PKCα translocation and CPI-17 phosphorylation. The translocation of PKCα from cytosol to membrane in guinea-pig left atrium cells was significantly increased by KMUP-3 (1 μM) (FIG. 12A). However, pretreatment with Y27632 (1-10 μM) insignificantly prevented KMUP-3-induced translocation of PKCα. Different expression ratios of cytosol/cytosol+membrane or membrane/cytosol+membrane at each concentration are shown in FIG. 12A. KMUP-3 concentration-dependently increased the phosphorylation of CPI-17 in electrically stimulated beating left guinea-pig atria (FIG. 12B).

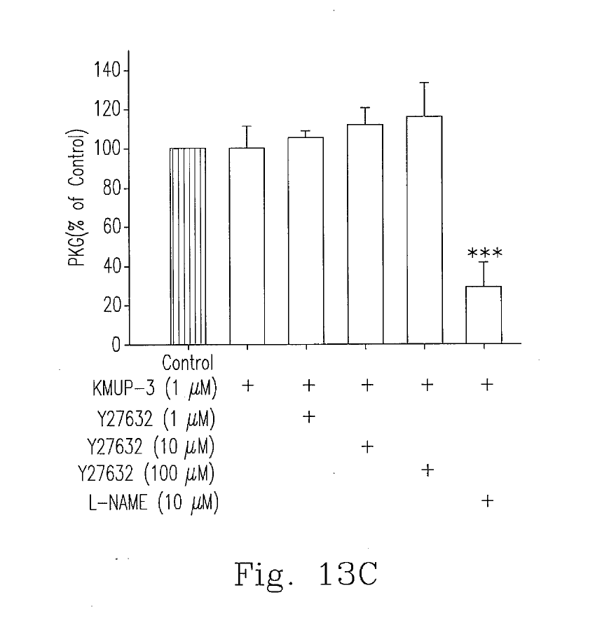

Pretreatment with Y27632 and L-NAME. As shown in FIG. 13 A-13 C, KMUP-3 significantly increased the expression of ROCKII (FIG. 13A) and PKA (FIG. 13B), but not PKG. KMUP-3-induced expression of PKA and ROCK II was inhibited by Y27632 (1-100 μM) but not PKG. Pretreatment with L-NAME reduced the expression of PKG (FIG. 13C).

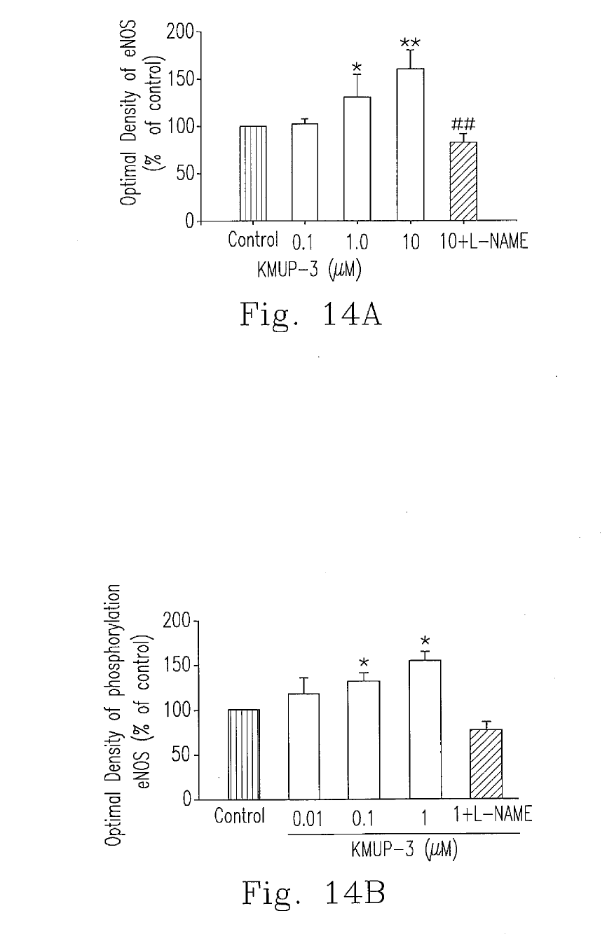

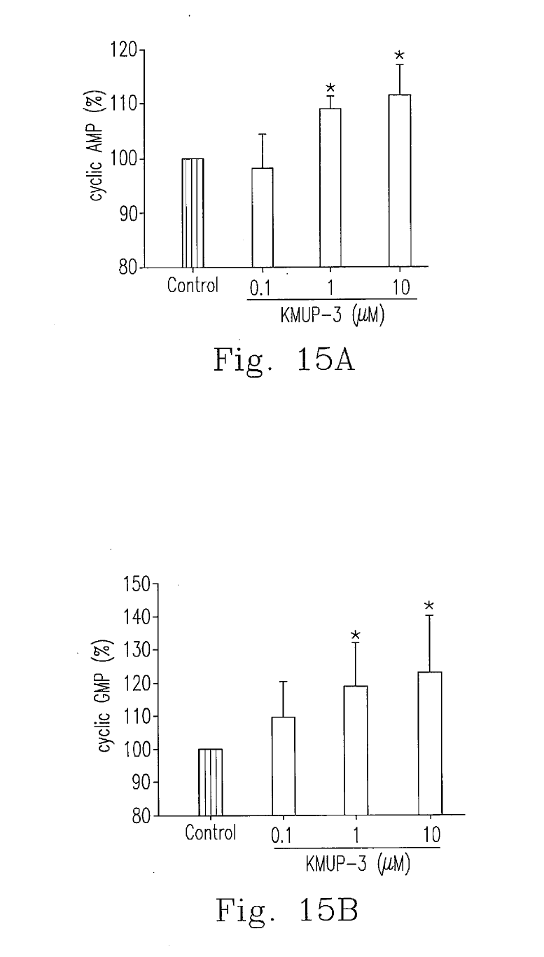

eNOS/p-NOS and cAMP/cGMP in neonatal myocytes. To determine the role of the NO/cGMP-pathway in heart, neonatal rat myocyte protein was extracted followed by Western blotting to measure the expression of eNOS, phosphorylated eNOS (p-eNOS), cAMP, and cGMP (FIG. 14 A-14 B and FIG. 15 A-15 B). KMUP-3 increased eNOS and p-eNOS in neonatal rat cardiomyocytes and the effect was reduced by L-NAME (100 μM)-pretreatment (FIG. 14A and FIG. 14B). KMUP-3 increased both cAMP and cGMP. The later was increased more than the former (FIG. 15A and FIG. 15B).

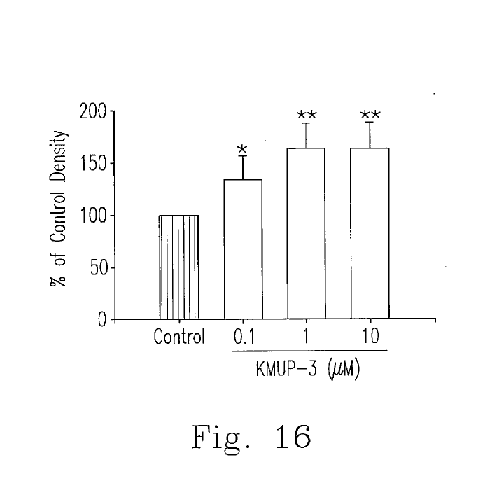

RhoA activation of neonatal myocytes. As shown in FIG. 16, Rho A activation, determined using an affinity precipitation assay which binds only the active GTP-bound form of Rho, was increased by KMUP-3.

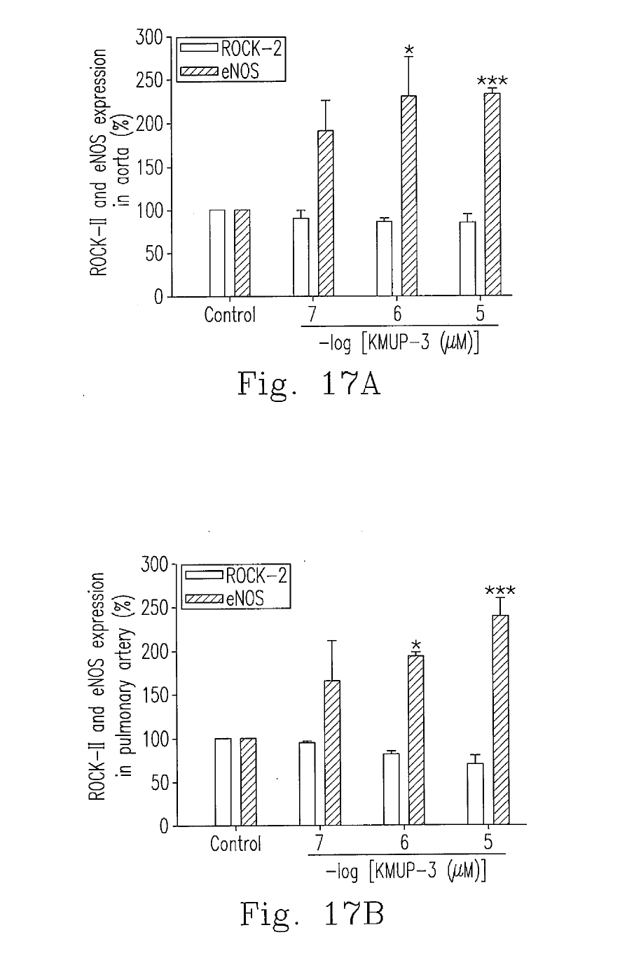

ROCKII expression and vascular resistance. To determine whether KMUP-3's ROCKII activity increased vascular resistance, aorta and pulmonary artery (PA) ring protein was extracted followed by Western blotting to measure the expression ROCKII and eNOS. We found that KMUP-3 did not increase vascular ROCKII, but did increase eNOS (FIG. 17 A-17 B).

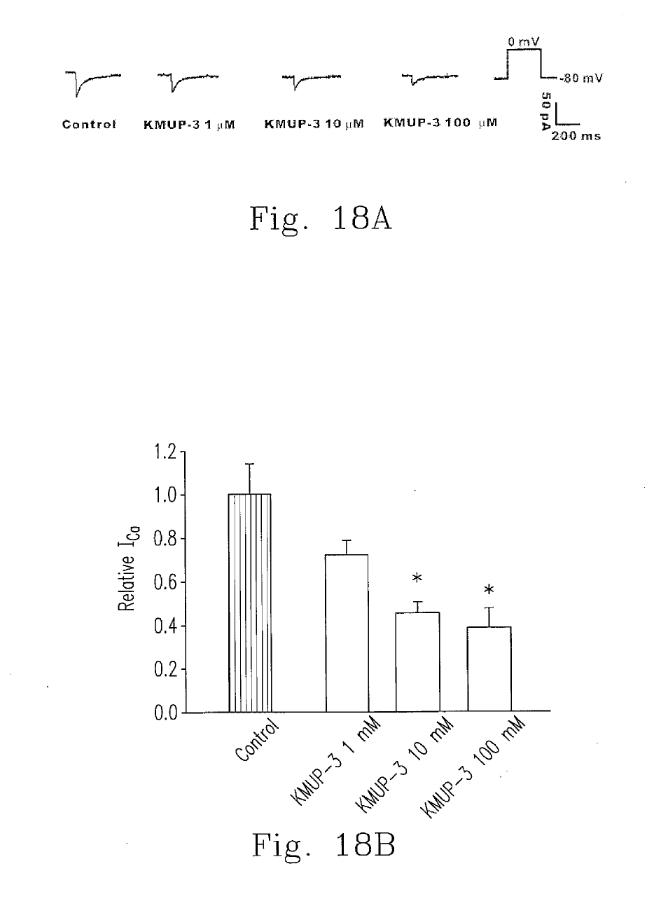

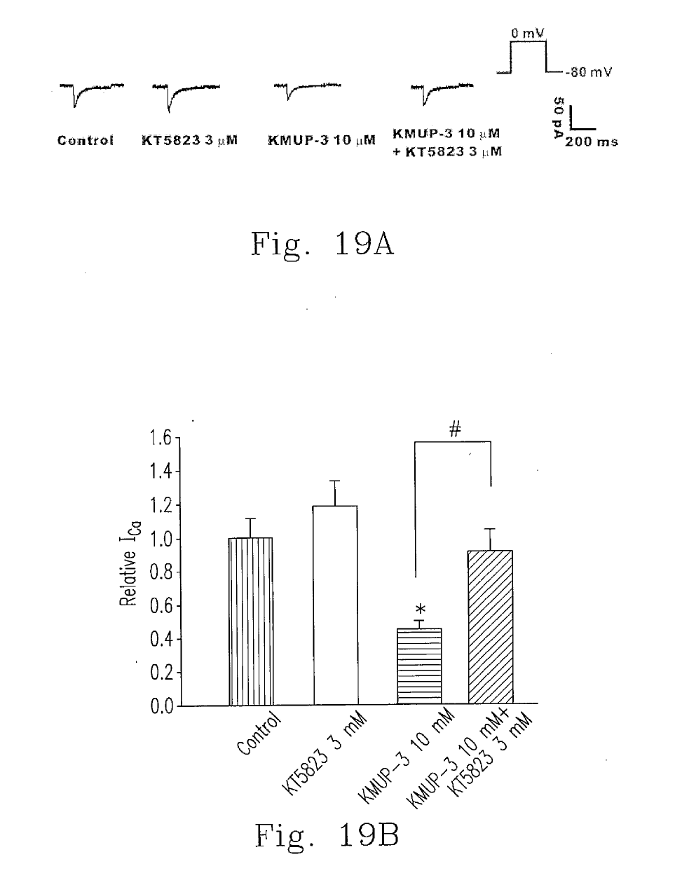

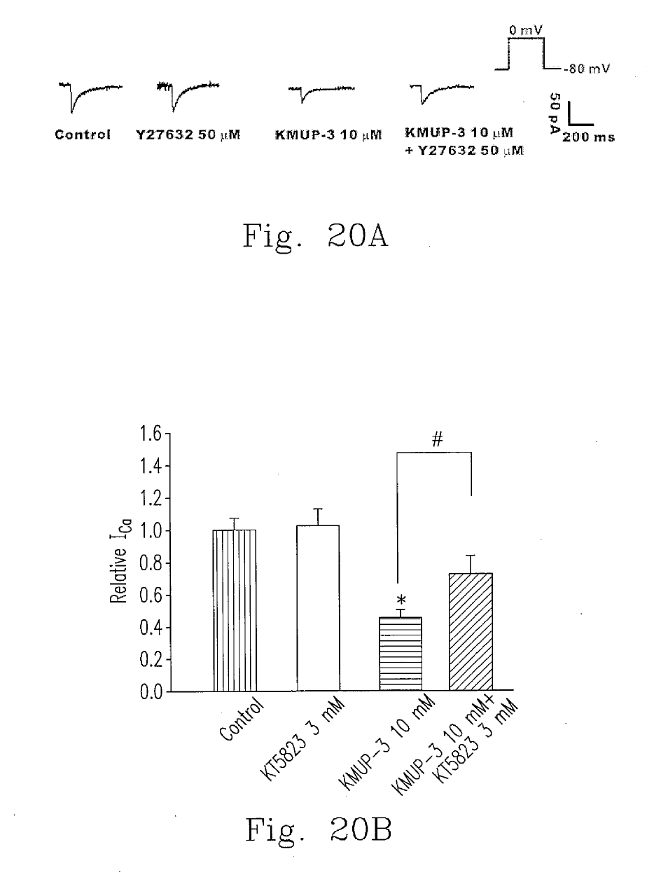

Cardiac Ca²⁺ entry blockade. In rat heart-derived H9c2 cells, KMUP-3 (1-100 μM) concentration-dependently reduced inward currents (FIG. 18A and FIG. 18B). As shown in FIG. 19 A-19 B and FIG. 20 A-20 B, combination of KMUP-3 (10 μM) with PKG antagonist KT5823 (3 μM) or ROCK inhibitor Y27642 (50 μM) restored the reduced currents.

Biological Experiments

Reagents

KMUP-3 and KMUP-3 amine salt compound was synthesized in our laboratory and dissolved in distilled water. SQ 22536 and L-NAME, KT5823 were purchased from Cayman Chemical Co. (Ann Arbor, Mich., U.S.A); Y27632, atropine, ketanserin and 7-imidazole, were all obtained from Sigma Chemical Co. (St. Louis, Mo., U.S.A.).

Anti-PKCα, anti-eNOS and anti-phospho-eNOS (Ser1177) antibody were obtained from BD transduction Laboratories (San Jose, Calif., U.S.A.) and Cell Signalling Technology (Beverly, Mass., U.S.A.), respectively. Anti-PKA_{RI} and Anti-PKG₁ antibodies were purchased from Calbiochem (San Diego, Calif., U.S.A). Anti-RhoA antibody was obtained from Santa Cruz Biotechnology (Santa Cruz, Calif., USA). Anti-ROCKII, anti-phospho CPI-17 and Anti-CPI-17 antibodies were obtained from Upstate Biotechnology (Lake Placid, N.Y., U.S.A.). All drugs and reagents were dissolved in distilled water, unless otherwise noted. KMUP-3 hydrochloride was dissolved in 5% glucose distill solution. Serial dilutions were made by distilled water.

Animals. Hartley guinea-pigs of either sex (350-500 g) and Wistar rats (200-250 g) were provided by the National Laboratory Animal Breeding and Research Center (Taipei, Taiwan) and housed under constant temperature and controlled illumination in Kaohsiung Medical University (KMU). Neonatal rats were obtained from copulation between healthy male and female rats. Food and water were available ad libitum. This study was approved by the Animal Care and Use Committee at KMU.

Hemodynamic Measurements.

Hemodynamic measurements were carried out in male Wistar rats weighting 300˜350 g, anesthetized with pentobarbital sodium (40 mg kg⁻¹, i.p.). In brief, polyethylene tubing (PE50) was placed in the trachea to keep the airway patent, and rats continued to breathe spontaneously. Subsequently, rats were used to record hemodynamic and cardiovascular function. Vascular catheters (PE50) were placed in the femoral arteries to measure mean arterial blood pressure (MABP) and heart rate (HR). Changes of MABP and HR caused by KMUP-3 or KMUP-3 amine salt were recorded from the femoral artery with a pressure transducer (Gould, Model P50, U.S.A.), connected with a Pressure Processor Amplifier (Gould, Model 13-4615-52, U.S.A.). A femoral vein was used for intravenous administration of KMUP-3 or KMUP-3 amine salt. A specialized transducer tip catheter was placed in the right carotid artery and advanced into the left ventricle of the heart for measurement of left ventricular pressure (LVSP) or contractility; dP/dt at 50 mm Hg, an index of cardiac contractility, was derived from the left ventricular pressure trace; post-hoc vascular resistance was calculated as mean arterial blood pressure/peripheral cardiac output (MABP/PCO). Body temperature was monitored throughout the experiment and maintained between 37 and 37.5° C. by using a heating pad.

Each dose of active drugs was administered as a 20-min infusion in a series of escalating doses dissolved in a 5% dextrose water vehicle. After termination of the high dose infusion, animals were observed for 20 min. KMUP-3 HCl or KMUP-3 amine salt were infused at 0.03, 0.05, and 0.075 μg/kg/20 min for the measurement of FABP, intraperitoneally injected at 0.03, 0.05 and 0.1 mg/kg for the measurement of MABP and HR and infused by intravenous injection for 2 min at 0.5, 1.0, 3.0 mg/kg dissolved in 5% dextrose water vehicle (2 ml) for the measurement of LV systolic pressure (LVSH), ABP, dP/dt, and HR. The primary hemodynamic variables were computed using commercial software and a signal processing workstation. Animals were randomly divided into one of six treatment or vehicle groups. After the completion of the surgical protocol, animals were allowed to stabilize for 1 h, and baseline data were collected at 5-min intervals for 30 min before the treatment.

Organ bath experiments and tissue procurement. Guinea-pigs were killed by a blow on the head. Their hearts were quickly excised and trimmed of excess tissues. The atrium was carefully removed and placed at room temperature (20˜25° C.) in a Krebs solution of the following composition (mM): NaCl 113, KCl 4.8, CaCl₂ 2.2, KH₂PO₄ 1.2, MgCl₂ 1.2, NaHCO₃ 25, dextrose 11.0; bubbled with a 95% O₂+5% CO₂ mixture, followed by separation of right and left atrium for subsequent experiments.

Spontaneously Beating Right Atrium.

Spontaneously beating right atrium was dissected from the heart and mounted in a 10 ml organ bath with one end fixed and the other end connected to a force displacement transducer (Grass, Model FT03). The frequency of contraction was measured on a separate channel by a tachometer (Coulbourn, Model S77-26) connected to a high-speed videograph (Coulbourn, AT L19-69). These experiments were carried out at 32.5° C. in Krebs solution. The atria strip was pre-stretched to a baseline tension of 0.5 g and equilibrated for 90 min in aerated (95% O₂+5% CO₂) Krebs solution before the experimental protocols were initiated. The atria were then allowed a 30-60 min washout period to re-stabilize. KMUP-3 or KMUP-3 amine salt (0.1, 1.0, 10, 100 μM) were then cumulatively added to the organ bath. To examine the possible involvement of NO and cAMP/cGMP in the action of KMUP-3 or KMUP-3 amine salt (0.1, 1.0, 10 μM) on spontaneously beating atria, the beating atria were pre-incubated with atropine (1 μM) and L-NAME (100 μM), respectively.

Electrically-Stimulated Beating Left Atrium.

Quiescent left atria free of connective tissues were dissected and mounted in an organ bath under a resting tension of 0.5 g. Organs were bathed at 32.5° C. in aerated Krebs solution and electrically stimulated with 1 Hz at 2-sec intervals via two platinum electrodes placed at either side. The positive inotropic effect was measured by determining the cumulative concentration-response curves in the presence of KMUP-3 or KMUP-3 amine salt (0.1, 1.0, 10, 100 μM). To examine the possible involvement of NO and cAMP/cGMP in the action of KMUP-3 or KMUP-3 amine salt on electrically-stimulated beating atria, pre-incubated with a ROCK inhibitor Y27632 (1.0 μM), a β-adrenoceptor blocker propranolol (1.0 μM), eNOS inhibitors L-NAME (100 μM), 7-nitroindazole (100 μM) and 5-HT inhibitor ketanserin (1.0 μM) was performed for 15 min prior to the addition of KMUP-3 or KMUP-3 amine salt to measure the development of beating force and heart rate for 10 min.

Protein extraction and Western blotting analysis. Briefly, after incubation with test agents in an organ bath, isolated beating atrium was frozen and pulverized to a fine powder at the temperature of liquid nitrogen. Atrium powders were then homogenized in protein extraction reagent (Pierce Biotechnology, Inc., Rockford, Ill.) by sonication. The samples were sonicated 10 sec three times and centrifuged at 13,000 rpm at 4° C. for 30 min. The protein concentrations of supernatants were determined by using bovine serum albumin as the standard. The cell extracts were then boiled in a ratio of 4:1 with sample buffer (Tris 100 mM, pH 6.8; glycerol 20%, Sodium dodecyl sulfate (SDS) 4% and bromophenol blue 0.2%). Electrophoresis was performed using 10% SDS-PAGE and transferred to nitrocellulose membranes (Millipore Corp., Billerica, Mass.). The membrane was blocked with Tris-buffered saline (TBS; 20 mM Tris and 137 mM NaCl, pH 7.6) containing 0.1% Tween 20 (TTBS) and 5% nonfat milk at room temperature for 1 h, washed with TTBS, and then incubated overnight at 4° C. in the appropriate primary antibody for PKA_{RI}, RhoA, ROCKII, p-CIP-17, CPI-17 and PKG_{1α1β}. The membranes were washed in TTBS before being incubated with horseradish peroxidase-conjugated antibody against mouse, goat, or rabbit IgG for 1 hr. Membranes were then washed in TTBS and developed with the enhanced chemiluminescence for the detection of the specific antigen. The intensity of the bands was measured by densitometry. eNOS or phosphorylated eNOS were extracted from cell culture of neonatal myocytes, PA ring and aortic ring (2-3 mm) of rats and measured by Western blotting analysis.

PK, ROCKII and CIP-17 expression and pretreatment of beating left atrium. To examine whether KMUP-3 or KMUP-3 amine salt affects the protein expression in beating atrium, PKA, ROCKII, PKG and phosphorylated CIP-17 was determined by Western blotting, respectively. To examine whether KMUP-3 or KMUP-3 amine salt affected cross-talk among ROCK and protein kinase PKA, PKCα and PKG, the ROCK inhibitor Y27632 (0.1, 1, 10, 100 μM) or eNOS inhibitor L-NAME (10 μM) was added to a beating atrium in the organ bath, respectively, for 15 min, followed by protein extraction.

PKCα Translocation of Beating Left Atrium.

To determine the effect of KMUP-3 or KMUP-3 amine salt on PKCa translocation, isolated left beating atrium was incubated with KMUP-3 or KMUP-3 amine salt for 30 minutes (0.1-10 μM). In brief, frozen left atrium was pulverized to a fine powder at the temperature of liquid nitrogen for preparation of cytosolic and membrane fractions. Isolated left atrium tissues were then homogenized by a sonicator (XL-2020, Heat Systems Ultrasonic, Plainview, N.Y., USA) at 4 in 1 ml of homogenization buffer containing 20 mM Tris-HCl (pH 7.5), 1 mM dithiothreitol (DTT), 5 mM EGTA, 2 mM EDTA, 0.5 mM PMSF, 20 μM leupeptin and 20 μM aprotinin. The cytosol fractions were obtained by collecting the supernatants after centrifuging the cells at 100,000×g for 45 minutes at 4. The pellets were resonicated in the homogenizing buffer with 1% Triton X-100, centrifuged again at 100,000×g for 45 minutes at 4, and the supernatants collected as membrane fractions. The extracts for cytosol and membrane fractions were denatured by heating at for 10 minutes in sampling buffer, and an aliquot containing 20 μg of total protein was subjected to a 10% SDS-polyacrylamide gel electrophoresis. After migration, proteins were transferred onto polyvinylidene difluoroide (PVDF) transfer membranes (Millipore), and the membranes were successively incubated at room temperature with 5% (w/v) nonfat dry milk in TBS for 1 hour, and then incubated with primary antibody at 4 overnight. Protein was detected with horseradish peroxidase (HRP) conjugated secondary antibody (BD Transduction Laboratories). At the end of the incubation, the membranes were extensively washed with TBS. The immunoreactive bands were detected by chemiluminescence (ECL) reagents (PerkinElmer Life Sciences, Inc).

RhoA Activation of Neonatal Myocytes.

RhoA activation was determined using an affinity precipitation assay, which binds only the active GTP-bound form of Rho. In brief, sub-confluent neonatal rat myocytes were grown to approximately 85-90% confluence, cells were exposed to KMUP-3 or KMUP-3 amine salt with 10% FBS for 90 min at 37 before addition of lysis buffer (25 mM HEPES, pH 7.5, 150 mM NaCl, 1% Igepal CA-630, 10 mM MgCl₂, 1 mM EDTA and 10% glycerol, 1 Ag/ml aprotinin, 10 Ag/ml leupeptin and 1 mmol/l Na₃PO₄) for 15 min at 4, cell lysates were clarified by centrifugation at 13000 g at 4 for 10 min, and equal volume of lysates were incubated with agarose conjugated rhotekin-RBD for 45 min at 4, then washed three time with lysis buffer. Agarose beads were boiled in SDS-PAGE sample buffer to release active RhoA in samples were resolved on a 12% polyacrylamide gel followed by immunoblotting with anti-Rho A (clone 55).

Measurement of Ca²⁺ Entry in H9c2 Cells.

To measure the I_{Ca} through L-type Ca²⁺ channels, whole cell patch-clamp electrophysiology was used in rat heart-derived H9c2 cells as previously described. In brief, H9c2 cells were placed in a recording dish and perfused with a bath solution containing (in mM): 135 tetraethylammonium (TEA)-Cl, 1.8 CaCl₂, 2 MgCl₂, 10 glucose, and 10 HEPES (pH 7.4, Tris). A recording electrode was pulled from borosilicate glass (resistance: 4-7 MW), and the pipette was coated with sticky wax close to the tip to reduce capacitance, backfilled with pipette solution containing (in mM): 140 CsCl, 1 EGTA, 1 MgCl₂, 5 Na₂ATP, and 5 HEPES (pH 7.2, Tris) and gently lowered onto a smooth muscle cell. Negative pressure was briefly applied to rupture the membrane and a gigaohm seal was obtained.

H9c2 cells were clamped at −80 mV with step depolarizations (300 ms) from −80 to 0 mV to evoke whole-cell I_{Ca}. Voltage clamped cells were equilibrated for 15 min prior to experimentation. Membrane currents were recorded on an Axopatch 700A amplifier (Axon Instruments, Union City, Calif.), filtered at 1 kHz using a low-pass Bessel filter, digitized at 5 kHz and stored on a computer for subsequent analysis with Clampfit 9.0. Following equilibration, I_{Ca} was monitored in the presence and absence of KMUP-3 or KMUP-3 amine salt (1, 10, 100 μM). To ascertain whether PKG or ROCK signaling was involved in the KMUP-3-induced decreases in I_{Ca}, H9c2 cells were incubated for 15 min with the combination of KMUP-3 or KMUP-3 amine salt (10 μM) and PKG antagonist KT5823 (3 μM) or ROCK inhibitor Y-27632 (50 μM). All electrical recordings were performed at room temperature.

Statistical Evaluation.

All data are expressed as the mean±S.E. Statistical differences were determined by independent and paired Student's t-test in unpaired and paired samples, respectively. Whenever a control group was compared with more than one treated group, one-way or two-way ANOVA was used. When ANOVA manifested a statistical difference, results were further analyzed with Dunnett's or Tukey's test. A probability value (p-value) less than 0.05 was considered to be significant. Data analysis and figure plotting were done with the aid of SigmaPlot software (Version 8.0, Chicago, Ill., U.S.A.) and SigmaStat (Version 2.03, Chicago, Ill., U.S.A.) run on an IBM compatible computer.

### Example 1

### Preparation of KMUP-3-Polylactic Acid Salt

2-Chloroethyl theophylline (8.3 g), NaOH (8.3 g) and 4-nitro-benzenepiperazin (8.3 g) are dissolved in hydrous ethanol (10 mL), and then heated under reflux for 3 h. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50 to 60, to make a saturated solution (pH 1.2). The saturated solution was sequentially treated, decolorized with activated charcoal, filtered, deposited overnight and filtered to obtain KMUP-3 HCl with a white crystal. Dissolved sodium polylactic acid (20 g) in 40 ml water to make 5% solution, after add KMUP-3 HCl (16 g) then heated under 50 for 10 min. Methanol (20 mL) is added thereinto under room temperature, and the solution is crystallized over night, after filtrated to obtain KMUP-3-polylactic acid salt (31.4 g).

### Example 2

### Preparation of KMUP-3-Polyglutamate Salt

Polyglutamate (2.5 g) is stirred in NaOH (10 mL) solution.

KMUP-3 (8.3 g) is dissolved in a mixture of ethanol (10 mL) and polyglutamate solution. The solution is reacted at 50° C. for 20 min, the methanol is added thereinto under room temperature, and the solution is incubated over night for crystallization and filtrated to obtain KMUP-3-polyglutamate salt (7.4 g).

### Example 3

### Preparation of KMUP-3-Hyaluronic Salt

KMUP-3 (8 g) is dissolved in a mixture of ethanol (10 mL) and hyaluronic acid (3.5 g). The solution is reacted at 50° C. for 20 min, the methanol is added thereinto under room temperature, and the solution is incubated over night for crystallization and filtrated to obtain KMUP-3-hyaluronic salt (10.6 g).

### Example 4

### Preparation of KMUP-3-Alginate-Poly-Lysine-Alginate Salt

KMUP-3 (8.3 g) is dissolved in a mixture of ethanol (10 mL) and alginate-poly-lysine-alginate (12.5 g). The solution is reacted at 50° C. for 20 min, the methanol is added thereinto under room temperature, and the solution is incubated over night for crystallization and filtrated to obtain KMUP-3-alginate-poly-lysine-alginate salt (20.4 g).

### Example 5

### Preparation of the Composition in Tablet

Tablets are prepared using standard mixing and formation techniques as described in U.S. Pat. No. 5,358,941, to Bechard et al., issued Oct. 25, 1994, which is incorporated by reference herein in its entirety.

| | |
|---|---|
| KMUP-3-polyglutamate salt | 1.05 g |
| Lactose | qs |
| Corn starch | qs |

### Embodiments

1. A inhibiting heart failure disease pharmaceutical composition, comprising:
an effective amount of a KMUP-3 amine salt represented by a structure being formula (I),

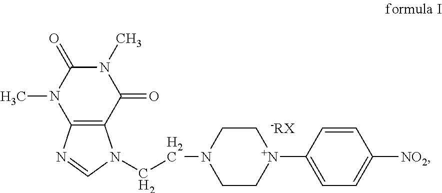

wherein the RX contains a carboxylic group donated from one of a sodium carboxyl methylcellulose (sodium CMC), a γ-polyglutamic acid derivative and a co-polymer salt, and the RX⁻ is an anion form of the carboxylic group; and
a pharmaceutically accepted carrier.
2. A improving cardiac output dose-dependently under Ca²⁺ entry protection pharmaceutical composition, comprising:
an effective amount of a KMUP-3 amine salt represented by a structure being formula (I),

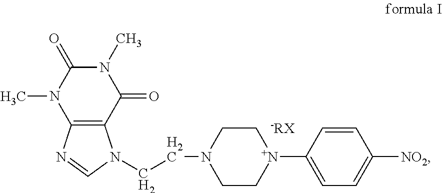

wherein the RX contains a carboxylic group donated from one of a sodium CMC, a γ-polyglutamic acid derivative and a co-polymer salt, and the RX⁻ is an anion form of the carboxylic group; and
a pharmaceutically accepted carrier.
3. A inhibiting heart failure disease complex compound, comprising:
an effective amount of a KMUP-3 amine salt represented by a structure being formula (I),

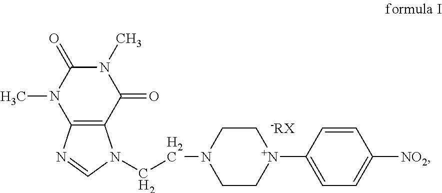

wherein RX contains a carboxylic group donated from one selected from a group consisting of a sodium CMC, a γ-polyglutamic acid derivative and co-polymer salt; and
RX⁻ is an anion form of the carboxylic group.
4. A improving cardiac output dose-dependently under Ca²⁺ entry protection complex compound, comprising:
an effective amount of a KMUP-3 amine salt represented by a structure being formula (I),

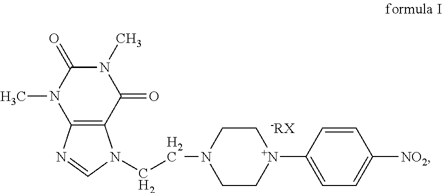

wherein RX contains a carboxylic group donated from one selected from a group consisting of a sodium CMC, a γ-polyglutamic acid derivative and Co-polymer salt; and
RX⁻ is an anion form of the carboxylic group.
5. A compound as above embodiments, wherein the RX is a γ-polyglutamic acid derivative being one of a sodium polyglutamate, an alginate-poly-lysine-alginate (APA) and a calcium polyglutamate-alginate sodium.
6. A compound as above embodiments, wherein the RX is a Co-polymer salts includes one selected from a group consisting of a hyaluronic acid, a polyacrylic acid, a dextran sulfate, a heparan sulfate, a polylactic acid (PLA), a polylactic acid sodium (PLA sodium) and a polyglycolic acid sodium (PGA sodium).
7. A method of providing a medical effect for inhibiting heart failure disease, comprising steps of:
providing a subject in need thereof; and
administering an effective amount of pharmaceutical composition of a KMUP-3 amine salt as above embodiments to the subject in need thereof.
8. A method of providing a medical effect for improving cardiac output dose-dependently under Ca²⁺ entry protection, comprising steps of:
providing a subject in need thereof; and
administering an effective amount of KMUP-3 amine salt above embodiments to the subject in need thereof.
9. A method of providing a medical effect for inhibiting heart failure disease, comprising steps of:
providing a subject in need thereof; and
administering an effective amount of pharmaceutical composition of a KMUP-3 amine salt as above embodiments to the subject in need thereof.
10. A method of providing a medical effect for improving cardiac output dose-dependently under Ca²⁺ entry protection, the method comprising steps of:
providing a subject in need thereof; and
administering an effective amount of pharmaceutical composition of above embodiments to the subject in need thereof.
11. A method of providing a medical effect for inhibiting heart failure disease, comprising steps of:
providing a subject in need thereof; and
administering an effective amount of pharmaceutical composition of a KMUP-3 amine salt as above embodiments to the subject in need thereof.
12. A method of providing a medical effect for inhibiting heart failure disease, comprising steps of:
providing a subject in need thereof; and
administering an effective amount of pharmaceutical composition of a KMUP-3 amine salt as above embodiments to the subject in need thereof.
13. A method of providing a medical effect for improving cardiac output dose-dependently under Ca²⁺ entry protection, comprising steps of: providing a subject in need thereof; and
administering an effective amount of pharmaceutical composition of a KMUP-3 amine salt as above embodiments to the subject in need thereof.
14. A method of providing a medical effect for inhibiting heart failure disease, providing a subject in need thereof; and
administering an effective amount of pharmaceutical composition of a KMUP-3 amine salt as above embodiments to the subject in need thereof.
15. A method of providing a medical effect for improving cardiac output dose-dependently under Ca²⁺ entry protection, comprising steps of: providing a subject in need thereof; and
administering an effective amount of pharmaceutical composition of a KMUP-3 amine salt as above embodiments to the subject in need thereof.
16. A method as above embodiments, wherein the KMUP-3 amine salt is one of a KMUP-3-CMC, a KMUP-3-γ-polyglutamic salt and KMUP-3-co-polymer salt.
17. A method as above embodiments, wherein the γ-polyglutamic acid derivative is one of a sodium polyglutamate, an alginate-poly-lysine-alginate (APA) and a calcium polyglutamate-alginate sodium.
18. A method as above embodiments, wherein the co-polymer salts includes one selected from a group consisting of a hyaluronic acid, a polyacrylic acid, a dextran sulfate, a heparan sulfate, a polylactic acid (PLA), a polylactic acid sodium (PLA sodium) and a polyglycolic acid sodium (PGA sodium).
19. A complex compound, comprising a KMUP-3 amine salt represented by a structure being formula (I),

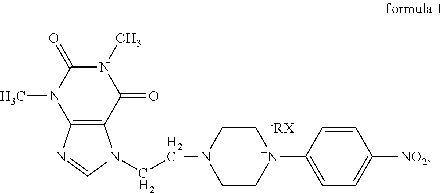

wherein the RX is one of a sodium CMC, a γ-polyglutamic acid derivative and a co-polymer salt, each of which has a carboxylic group, and the RX⁻ has an electron donated form the carboxylic group.
20. A cardiac output increasing-pharmaceutical composition, comprising:
   an effective amount of a KMUP-3 amine salt as above embodiments; and
   a pharmaceutically accepted carrier.
21. A pharmaceutical composition as claimed in claim 15, wherein the KMUP-3-amine salt is synthesized by a KMUP-3 and a carboxylic group donated from one of a sodium CMC, a γ-polyglutamic acid derivative and a co-polymer salt.
A method of providing a medical effect for improving chronic cardiac insufficiency, comprising steps of:
providing a subject in need thereof; and
administering an effective amount of pharmaceutical composition of a KMUP-3 amine salt as above embodiments to the subject in need thereof.

### REFERENCE

1. Endoh M. Could Ca²⁺ sensitizers rescue patients from chronic congestive heart failure? Br J Pharmacol 2007; 150: 826-828.

What is claimed is:

1. A complex compound, comprising a KMUP-3 amine salt represented by a structure being formula (I), 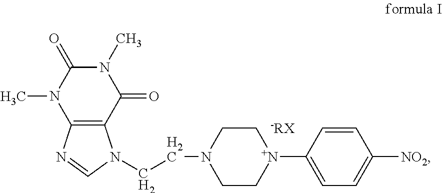
wherein the RX is one of a sodium carboxyl methylcellulose (sodium CMC), a γ-polyglutamic acid derivative and a co-polymer salt, each of which has a carboxylic group, and the RX⁻ has an electron donated form the carboxylic group.

2. A compound as claimed in claim 1, wherein the RX is the γ-polyglutamic acid derivative being one of a sodium polyglutamate, an alginate-poly-lysine-alginate (APA) and a calcium polyglutamate-alginate sodium.

3. A compound as claimed in claim 1, wherein the RX is a co-polymer salt including one selected from a group consisting of a hyaluronic acid, a polyacrylic acid, a dextran sulfate, a heparan sulfate, a polylactic acid (PLA), a polylactic acid sodium (PLA sodium) and a polyglycolic acid sodium (PGA sodium).

4. A compound as claimed in claim 1, wherein the KMUP-3-amine salt is a 7-[2-[4-(4-nitrobenzene)piperazinyl]ethyl]-1,3-dimethylxanthine-amine salt.

5. A heart failure disease inhibiting-pharmaceutical composition, comprising:
an effective amount of a KMUP-3-amine salt as claimed in claim 1; and
a pharmaceutically accepted carrier.

6. A pharmaceutical composition as claimed in claim 5, wherein the KMUP-3-amine salt is synthesized by a KMUP-3 and one of a sodium carboxyl methylcellulose (sodium CMC), a γ-polyglutamic acid derivative and a co-polymer salt.

7. A pharmaceutical composition as claimed in claim 6, wherein the co-polymer salt includes one selected from a group consisting of a hyaluronic acid, a polyacrylic acid, a dextran sulfate, a heparan sulfate, a polylactic acid (PLA), a polylactic acid sodium (PLA sodium) and a polyglycolic acid sodium (PGA sodium).

8. A pharmaceutical composition as claimed in claim 6, wherein the γ-polyglutamic acid derivative is one of a sodium polyglutamate, an alginate-poly-lysine-alginate (APA) and a calcium polyglutamate-alginate sodium.

9. A pharmaceutical composition as claimed in claim 6, wherein the KMUP-3-amine salt is a 7-[2-[4-(4-nitrobenzene)piperazinyl]ethyl]-1,3-dimethylxanthine-amine salt.

10. A method of providing a medical effect for inhibiting heart failure disease, comprising steps of:
providing a subject in need thereof; and
administering an effective amount of pharmaceutical composition of a KMUP-3 amine salt as claimed in claim 1 to the subject in need thereof.

11. A method as claimed in claim 10, wherein the KMUP-3-amine salt is synthesized by a KMUP-3 and one of a sodium carboxyl methylcellulose (sodium CMC), a γ-polyglutamic acid derivative and a co-polymer salt.

12. A method as claimed in claim 11, wherein the co-polymer salt includes one selected from a group consisting of a hyaluronic acid, a polyacrylic acid, a dextran sulfate, a heparan sulfate, a polylactic acid (PLA), a polylactic acid sodium (PLA sodium) and a polyglycolic acid sodium (PGA sodium).

13. A method as claimed in claim 11, wherein the RX is the γ-polyglutamic acid derivative being one of a sodium polyglutamate, an alginate-poly-lysine-alginate (APA) and a calcium polyglutamate-alginate sodium.

14. A method as claimed in claim 11, wherein the KMUP-3-amine salt is a 7-[2-[4-(4-nitrobenzene)piperazinyl]ethyl]-1,3-dimethylxanthine-amine salt.

15. A cardiac output increasing-pharmaceutical composition, comprising:
an effective amount of a KMUP-3 amine salt as claimed in claim 1; and
a pharmaceutically accepted carrier.

16. A pharmaceutical composition as claimed in claim 15, wherein the KMUP-3-amine salt is synthesized by a KMUP-3 and a carboxylic group donated from one of a sodium carboxyl methylcellulose (sodium CMC), a γ-polyglutamic acid derivative and a co-polymer salt.

17. A pharmaceutical composition as claimed in claim 16, wherein the γ-polyglutamic acid derivative is one of a sodium polyglutamate, an alginate-poly-lysine-alginate (APA) and a calcium polyglutamate-alginate sodium.

18. A pharmaceutical composition as claimed in claim 16, wherein the co-polymer salt includes one selected from a group consisting of a hyaluronic acid, a polyacrylic acid, a dextran sulfate, a heparan sulfate, a polylactic acid (PLA), a polylactic acid sodium (PLA sodium) and a polyglycolic acid sodium (PGA sodium).

19. A method of providing a medical effect for improving cardiac output, comprising steps of:
providing a subject in need thereof; and
administering an effective amount of pharmaceutical composition of a KMUP-3 amine salt as claimed in claim 15 to the subject in need thereof.

20. A method as claimed in claim 19, wherein the KMUP-3-amine salt is synthesized by a KMUP-3 and a carboxylic group donated from one of a sodium carboxyl methylcellulose (sodium CMC), a γ-polyglutamic acid derivative and a co-polymer salt.
